(19) United States
(12) Patent Application Publication
(10) Pub. No.: US 2025/0352274 A1
(43) Pub. Date: Nov. 20, 2025

(54) TRACKING AND MANEUVERING USING AN ELECTROMAGNETIC SENSOR

(71) Applicant: Northern Digital Inc., Waterloo (CA)

(72) Inventors: Nikolai Mikuszeit, Radolfzell (DE); Marco Ewen, Hohenfels (DE)

(21) Appl. No.: 19/058,792

(22) Filed: Feb. 20, 2025

Related U.S. Application Data
(60) Provisional application No. 63/556,095, filed on Feb. 21, 2024.

(51) Int. Cl. A61B 34/20; A61B 34/00

(52) U.S. Cl. CPC A61B 34/20; A61B 34/73; A61B 2034/2051

(57) ABSTRACT

A device comprising an insertable structure usable in a surgical theater; one or more permanent magnets positioned at respective locations in the insertable structure, and one or more electromagnetic sensors. Each of the one or more permanent magnets has a magnetization axis. At least a portion of each of the one or more electromagnetic sensors is wrapped around one of the one or more permanent magnets such that a coil axis of the respective electromagnetic sensor substantially aligns with the magnetization axis of the corresponding permanent magnet. The device is configured to be located and have movement initiated according to two different external magnetic fields controlled by a computer system.

### CLAIM OF PRIORITY

This application claims priority under 35 USC § 119 (e) to U.S. Patent Application Ser. No. 63/556,095, filed on Feb. 21, 2024, the entire contents of which are hereby incorporated by reference.

### TECHNICAL FIELD

This disclosure relates to positioning and maneuvering using an electromagnetic (EM) sensor.

### BACKGROUND

Electromagnetic Tracking (EMT) systems are used to aid in locating instruments and anatomy in medical procedures. These systems utilize a magnetic transmitter in proximity to one or more magnetic sensors. By receiving transmissions from the magnetic transmitter, the electromagnetic sensor produces signals that can be employed for tracking instruments or other objects associated with medical procedures. One or more sensors (electromagnetic sensors, optical sensors, etc.) can be spatially located relative to the transmitter and may be equipped for EM and/or optical tracking.

### SUMMARY

Systems (e.g., EMT systems) that identify pose (i.e., position and orientation) information for instruments used in medical procedures can include system components to make measurements with respect to patient anatomy. In this document, the above-noted systems can include components for achieving tracking purposes (e.g., tracking instruments, system components, etc.) during medical procedures, but also configured to maneuver (e.g., control the guidance and movement, etc.) instruments, system components, etc. while they are tracked.

Medical procedures using the herein-described devices, systems, etc. can span many domains and applications-including surgical interventions, diagnostic procedures, imaging procedures, radiation treatment, etc. Examples of medical procedures can include endoscopy, vascular catheterization, GI and pulmonary studies, etc.

In some implementations, a device including multiple electromagnetic (EM) sensors can be combined with magnets for tracking the device and controlling the maneuvering of the device by utilizing different types of magnetic fields (e.g., an alternating current (AC) magnetic field or a direct current (DC) magnetic field). For example, a system can locate or track a device (e.g., a medical device, medical tool, etc.) by using an AC magnetic field to induce a voltage in EM sensors. The system can maneuver the device by controlling a movement or movements of a magnet (e.g., a soft magnet, hard magnet, etc.) using a DC magnetic field. In general, soft magnets can be considered as being easily magnetized and de-magnetized, while hard magnets typically retain their magnetization even in the absence of an external magnetic field.

Objects (e.g., devices, tools, etc.) that are tracked by systems for medical procedures can include devices that are uniquely identifiable and trackable by optical cameras, electromagnetic sensors that measure a change in a magnetic field produced by a magnetic field generator, combinations of cameras and sensors, etc. Systems with EM sensors can further include a permanent magnet for maneuvering objects used in medical procedures, after resolving undesired interactions between the AC magnetic field and the DC magnetic field (e.g., described with respect to FIGS. 1A, 1B, and 1C).

In some implementations, a trackable object can include a device, e.g., a medical device such as a catheter having a guidewire or a fiber optic line. The trackable object can be located or tracked by a computer system that determines poses of the trackable object (a catheter, a guidewire, or a fiber optic line, etc.). To determine a pose of the trackable object, the computer system measures poses of one or more EM sensors located in the trackable object in an AC magnetic field. For purposes of simplified illustration, the following specification is described generally in relation to a device (e.g., a catheter). However, it should be understood that the described techniques are applicable to a general trackable object.

In one aspect, the described technique relates to a device. The device includes an insertable structure usable in a surgical theater, one or more permanent magnets positioned at respective locations in the insertable structure, and one or more electromagnetic sensors. Each of the one or more permanent magnets has a magnetization axis. At least a portion of each of the one or more electromagnetic sensors is wrapped around one of the one or more permanent magnets such that a coil axis of the respective electromagnetic sensor substantially aligns with the magnetization axis of the corresponding permanent magnet. The device is configured to be located and have movement initiated according to two different external magnetic fields controlled by a computer system.

Embodiments can include one or any combination of two or more of the following features.

In some implementations, the two different external magnetic fields can be generated by at least one magnetic field generator. The two different external magnetic fields can include an alternating current (AC) magnetic field and a direct current (DC) magnetic field.

The device can be configured to be located by the computer system using the AC magnetic field, and the device can be configured to have the movement initiated by the computer system according to the DC magnetic field.

In some implementations, at least one of the one or more permanent magnets can define an internal channel.

The device can further a fiber optic line extending through the insertable structure and passing through the internal channel. In addition or alternatively, the device can further include a guidewire extending through the insertable structure and passing through the internal channel.

In some implementations, one electromagnetic sensor can have a solenoidal geometry that includes a plurality of windings that include one or more substantially orbital turns. The device can further include a magnetic motor, and at least one of the permanent magnets can be located inside the magnetic motor.

In addition, one permanent magnet can oscillate around an oscillating axis, and the coil axis of the electromagnetic sensor can substantially align with the oscillating axis. Moreover, each of the one or more permanent magnets can generate a magnetic field with a magnitude smaller than a magnetic saturation magnitude.

According to another aspect, combinable with the foregoing aspect, the described technique relates to a system, which includes a device and a computer system. The device includes an insertable structure usable in a surgical theater, one or more permanent magnets positioned at respective locations in the insertable structure, each of the one or more permanent magnets having a magnetization axis, and one or more electromagnetic sensors, where at least a portion of each of the one or more electromagnetic sensors is wrapped around one of the one or more permanent magnets such that a coil axis of the respective electromagnetic sensor substantially aligns with the magnetization axis of the corresponding permanent magnet. The computer system includes a memory, and a processor configured to generate a set of instructions that, once executed, control two different external magnetic fields for locating the device and initiating a movement of the device.

Embodiments can include one or any combination of two or more of the following features.

The two different external magnetic fields are generated by at least one magnetic field generator, and the two different external magnetic fields include an alternating current (AC) magnetic field and a direct current (DC) magnetic field. The device is configured to be located by the computer system using the AC magnetic field, and the device is configured to have the movement initiated by the computer system according to the DC magnetic field.

At least one of the one or more permanent magnets defines an internal channel. The device further includes a fiber optic line extending through the insertable structure and passing through the internal channel. The device further includes a guidewire extending through the insertable structure and passing through the internal channel.

One of the one or more electromagnetic sensors has a solenoidal geometry that includes a plurality of windings that include one or more substantially orbital turns.

The device includes a magnetic motor, and wherein at least one of the permanent magnets is located inside the magnetic motor.

One of the one or more permanent magnets oscillates around an oscillating axis, and wherein the coil axis of the electromagnetic sensor substantially aligns with the oscillating axis.

Each of the one or more permanent magnets generates a magnetic field with a magnitude smaller than a magnetic saturation magnitude.

Implementations may provide one or more of the following advantages. The described techniques can allow a system to accurately track a device (by determining the position, orientation, etc. of the device) and control the maneuvering of the device being used in a medical procedure. The techniques described herein incorporate multiple EM sensors that include permanent magnets to avoid undesired magnetic saturation that can reduce the effectiveness of EM sensors and potentially render EM sensors inoperable. By employing these techniques, devices can be accurately tracked using an external AC magnetic field and can efficiently have movements controlled by using an external DC magnetic field.

In some cases, the described techniques employ a fiber optic line, a guidewire, or both that extends through internal channels defined by the device. A fiber optic line can be used to improve the accuracy of tracking the device using EM sensors. A guidewire can be used as a guide for the placement of the device, e.g., a catheter.

The described techniques efficiently assist with managing the geometry and size of a device including one or more EM sensors. The described techniques generally relate to wrapping an EM coil around a permanent magnet, and such an arrangement might require a greater space in a device than an air coil (wrapping an EM coil around air). However, wrapping an EM coil around a permanent magnet using the described techniques does not necessarily require a greater space if the device already includes one or more permanent magnets. An EM coil can be readily wrapped around these permanent magnets without needing more space, which could lead to a need for a larger device or assembly. For example, the device can be a magnetic motor with one or more permanent magnets.

The details of one or more embodiments of the subject matter described herein are set forth in the accompanying drawings and the description below. Other features, objects, and advantages of the subject matter will be apparent from the description and drawings, and from the claims.

### DESCRIPTION OF DRAWINGS

FIG. 1A is a diagram of an example system.

FIG. 1B is a block diagram of components in a computing device of FIG. 1A and peripheral components coupled to the computing device.

FIG. 1C is a block diagram of components in a computing device of FIG. 1A peripheral components coupled to the computing device.

FIG. 2A shows a cross-sectional view of an example device that includes a catheter and electromagnetic sensors.

FIG. 2B shows a cross-sectional view of another example device that includes a catheter and electromagnetic sensors.

FIG. 2C shows a cross-sectional view of another example device that includes a catheter and electromagnetic sensors.

FIGS. 3A and 3B show examples of electromagnetic sensors.

FIG. 4 is a flowchart of operations executable by the system of FIG. 1A.

FIG. 5 shows a schematic diagram of an example computer system.

Like reference numbers and designations in the various drawings indicate like elements.

### DETAILED DESCRIPTION

A system can be used to track, locate, etc. one or more objects within medical contexts (e.g., objects used in medical procedures). For example, objects to be located can include a device, a tool, etc. used in a medical procedure, and such objects can include, for example, one or more types of medical equipment, a robotic arm, etc. The system can track an object by determining the pose (e.g., three-dimensional location, orientation, etc.) of the object and the tracked pose can be presented by the system to a medical professional (e.g., a surgeon) through a user interface (e.g., presented on a display of a computing device). Various types of functionalities can be provided by the tracking system; for example, data prepared by the system can be used for providing guidance in image-guided procedures. Use of such systems may allow for reduced reliance on other imaging modalities, such as fluoroscopy, which can expose the patient to the health risk of ionizing radiation. Such systems are described in U.S. application Ser. No. 17/877,117, entitled “TRACKING SYSTEM,” filed on Jul. 30, 2022, which is hereby incorporated by reference in its entirety.

In some implementations, the system used in medical contexts can be configured to maneuver one or more objects used in a medical procedure. For example, a system can maneuver a motion of an object by manipulating an external direct current (DC) magnetic field using a magnetic field generator to cause a magnet located in (or on) the object to move. Various types of magnets can be employed, for example, the magnet to be moved by the external DC magnetic field can be a soft magnet or a hard magnet. Soft magnets generally refer to a magnet that is easily magnetized or de-magnetized. Hard magnets can be considered magnets that, once magnetized, are resistant to external magnetic field changes. Hard magnets generally have a higher coercivity than soft magnets.

A system for tracking purposes (also referred to as an EMT or an EM tracking system hereinafter) can implement electromagnetic (EM) tracking functionality to track 5 degrees of freedom (DOFs) or 6 DOFs of a target object. A degree of freedom refers to an independent and mutually exclusive parameter or direction in which an object or system can move or vary. Generally, the EMT system includes a transmitter configured to generate an external magnetic field applied to one or more EM sensors located in the magnetic field. An EM sensor can include an EM coil wrapping around a core. In cases where the EM coil wraps around the air, it is also referred to as an air coil. The external magnetic field can include an alternating current (AC) magnetic field, a direct current (DC) magnetic field, AC and DC magnetic fields in combination, etc. A DC magnetic field refers to a magnetic field that generally remains constant in strength and direction over time, as opposed to an AC magnetic field, which periodically changes direction and magnitude. Typically, an external AC magnetic field is used for tracking purposes since the magnetic amplitude varies with time, and such a varying magnitude could induce voltage that could also vary in time in EM sensors. For example, an AC magnetic field can induce voltages in an EM sensor that contains a coil of conductive material (e.g., conductive wires). The EM sensor can provide sensor data representing the varying induced voltage to a computing system for tracking purposes.

One or more sensors having one or more EM coils that are in proximity to the generated EM field are configured to measure the characteristics (e.g., amplitude of an induced voltage, phase of an induced voltage) of the external AC magnetic field. The measured characteristics of the AC magnetic field depend upon the position and orientation of the EM sensors relative to the transmitter. For example, when the sensors are located at a particular position and orientation, the external magnetic field at that particular location may have particular characteristics (e.g., amplitude, phase, etc.). The EM sensors can measure the characteristics of the EM field and provide measured quantities (e.g., via sensor signals such as induced voltages) to a computing device (e.g., a computer system). Using information related to the external AC magnetic field and the EM sensor signals received from the sensors, the computing device can determine the position, orientation, etc. of the EM sensors. By employing this technique, the position, orientation, etc. of a medical device that contains the EM sensors can be identified and used by the computing device (e.g., the computing device can include a display to graphically represent the medical device, the sensor, registered medical images, etc.). Some tracking systems can also be equipped with fiber optic shape sensing (FOSS) capabilities. For example, while one or more EM sensors may be able to provide pose information for two discrete points (e.g., X-Y points in 3D space), FOSS can be used to ascertain shape information for a continuous segment (e.g., a segment of a catheter).

However, tracking and maneuvering devices using both soft magnets and external magnetic fields (both AC and DC magnetic fields) may saturate EM sensors and reduce the effectiveness of the EM sensors. This is generally because soft magnets (e.g., air coils) tend to saturate in the presence of an external DC magnetic field. Saturation generally refers to the state of a magnet when the magnet is exposed to a strong external magnetic field. A magnet can sometimes include a magnetic core to increase the strength of a voltage field or a magnetic field induced by an external magnetic field. In the saturation state, an increase in the external magnetic field would not proportionally increase the magnetization of the magnet. Thus, the total magnetic flux density in the magnet generally levels off, rendering EM sensors surrounding the cores inoperable for tracking the positions of a corresponding device.

In general, an EM sensor that includes one or more coils winding around a core can be used to determine a position in an external AC magnetic field based on EM signals in the EM sensor induced by an external magnetic field. The EM signals are produced by the EM sensor based partially on the magnetic flux of the external AC magnetic field passing through the winding of the EM sensor coil. Yet the EM signals are produced based mainly on the magnetization of the sensor coil induced by the external AC magnetic field. The amplitude or magnitude of the EM signals is substantially linear to the amplitude of the external AC magnetic field before the induced magnetization reaches the “saturation” state. The linear relationship between the induced magnetization and the external field ensures the tracking functionality of an EM sensor. The “saturation” magnitude generally depends on the properties of a sensor design. For example, a saturation magnitude can range from 20 μT to 50 μT. However, one should appreciate that other suitable saturation magnitudes are applicable due to particular design requirements. Since a soft magnet can be magnetized due to an external magnetic field, a soft magnet can reach the “saturation” state upon being exposed to a relatively strong magnetic field (e.g., a magnetic field introduced by a permanent magnet). In particular, a soft magnet would become saturated when it is exposed to a DC magnetic field with a magnitude a few orders greater than that of an external AC magnetic field. The AC magnetic field can introduce some magnetic flux to the sensor coil; however, typically, the induced field is unable to move the soft magnet totally away from the “saturation” state caused by the strong DC magnetic field. When the soft magnet is “saturated,” the magnetization stays substantially constant with time. Thus, the induced voltage on the EM sensor is merely generated by the magnetic flux through the coil by the external AC magnetic field—not by the induced magnetization anymore. The EM signals reduce considerably and may become barely measurable. Due to this condition, the EM sensor becomes nearly inoperable in terms of tracking a location in the AC magnetic field. If a magnet partially enters the “saturation” state, the magnet is still not suitable for tracking functionality.

The described techniques can resolve the above-noted “saturation” issue when tracking and moving a device using different external fields. More specifically, the described techniques can replace soft magnets with hard magnets, particularly by wrapping EM sensor coils around hard magnets. One or more EM sensor coils each are winded around a corresponding permanent magnet that allows the EM sensor coils to stay away from the “saturation” state. Although soft magnets (or soft magnetic cores) are preferable to occupy less space since the coils can have small cross-sectional areas, winding an EM sensor coil around a permanent magnet does not necessarily require greater space or render a larger device. Rather, coils can be winded around permanent magnetics already included in a device or tool. Indeed, it is not uncommon to see devices including one or more permanent magnets. For example, a magnetic motor can include one or more permanent magnets for steering the magnetic motor. This way, the described techniques can improve the tracking accuracy and efficiency using EM sensors in both AC and DC magnetic fields.

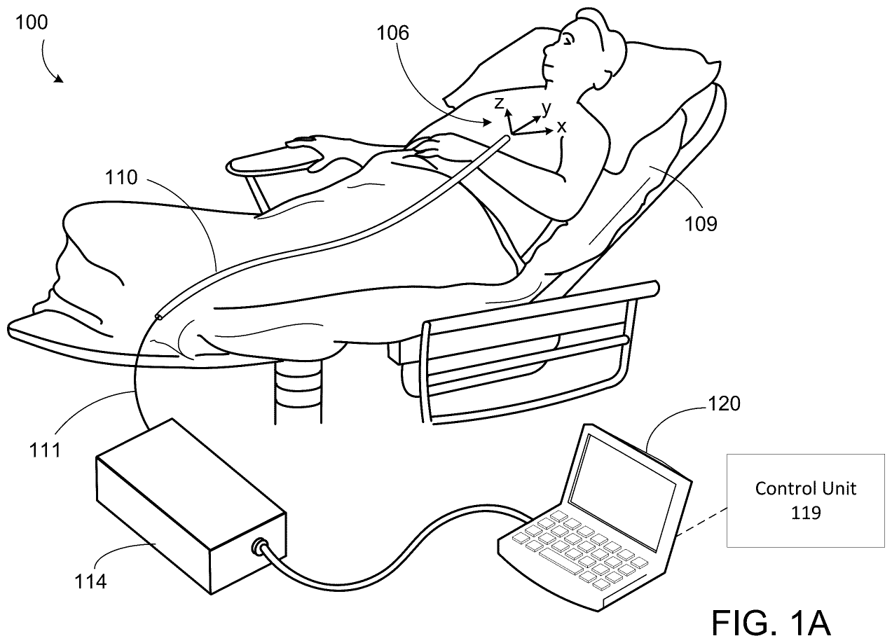

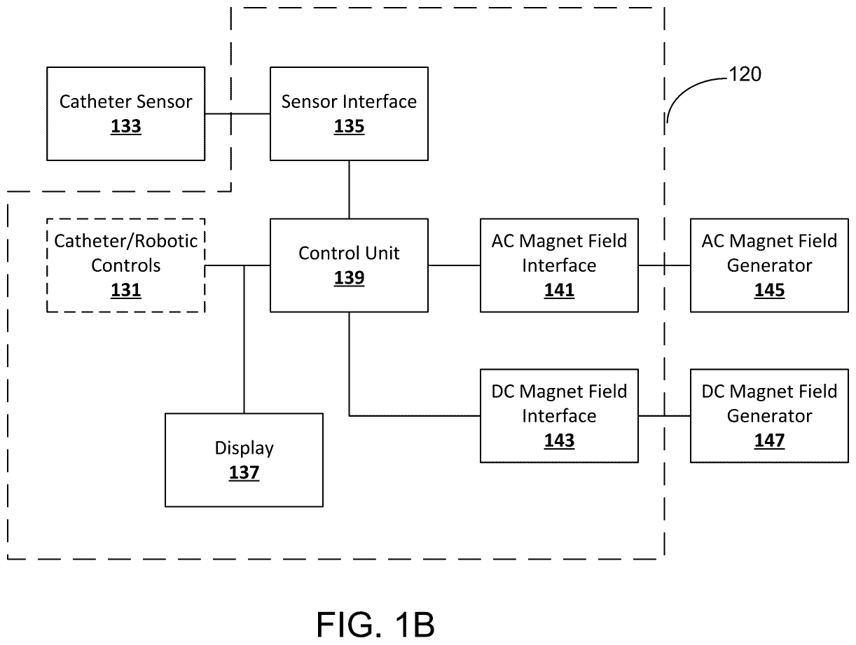

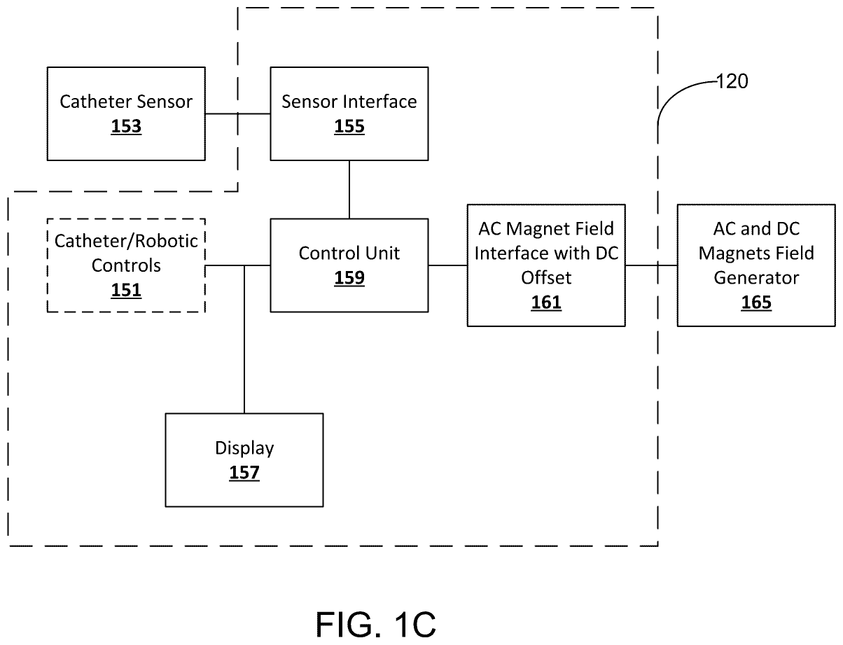

FIG. 1A shows an example system 100 that is implemented in the surgical environment (e.g., a surgical theater). System 100 is configured to determine the location of one or more electromagnetic sensors, such as one or more sensors embedded in a catheter or another structure (e.g., surgical equipment such as a scalpel, probe, guidewire, etc.) located within a patient. The electromagnetic tracking techniques employed for tracking medical devices, for example, may be similar to those described in U.S. Patent Application Publication No. 2013/683,703, entitled “Tracking a Guidewire,” filed on Nov. 21, 2012, which is hereby incorporated by reference in its entirety. The electromagnetic tracking techniques described herein can employ a computing device (e.g., a computer system), a transmitter excitation component, and a receiving component. Under computer control, a multi-axis transmitter assembly can have each of its axes (e.g., an X-axis or Y-axis in an XYZ 3-dimensional coordinate frame) energized by drive electronics (e.g., DC drive electronics or DC magnetic field generator, AC drive electronics or AC magnetic generator, etc.) to transmit external magnetic fields. The external magnetic fields can take forms of waveforms (e.g., symmetrical, sequentially excited, non-overlapping square DC-based waveforms). These waveforms are received through the air or tissue by one or more sensors that convey these signals to signal-processing electronics within the electromagnetic tracking system electronics. The computer in the electromagnetic tracking system electronics can execute various processing operations; for example, it can measure the rising edge and steady state of each axis' sequential waveform (e.g., using an integrator) so that a result (e.g., an integrated result) may be measured at the end of the steady-state period. The computer can further control the transmitter drive electronics to operate the transmitter and receive signals from the signal processing electronics for one or more processes (e.g., the signal integration process), the end result being a calculation of the sensor's position and orientation in three-dimensional space.

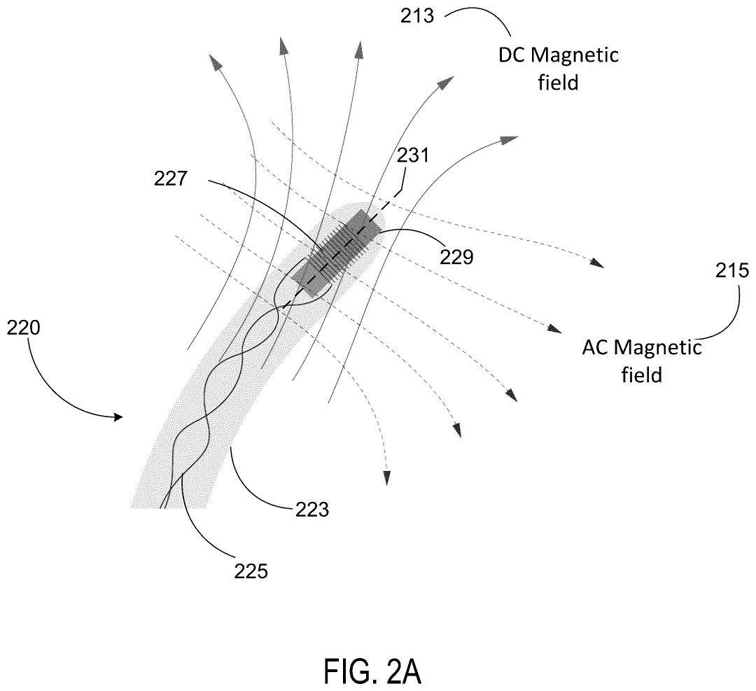

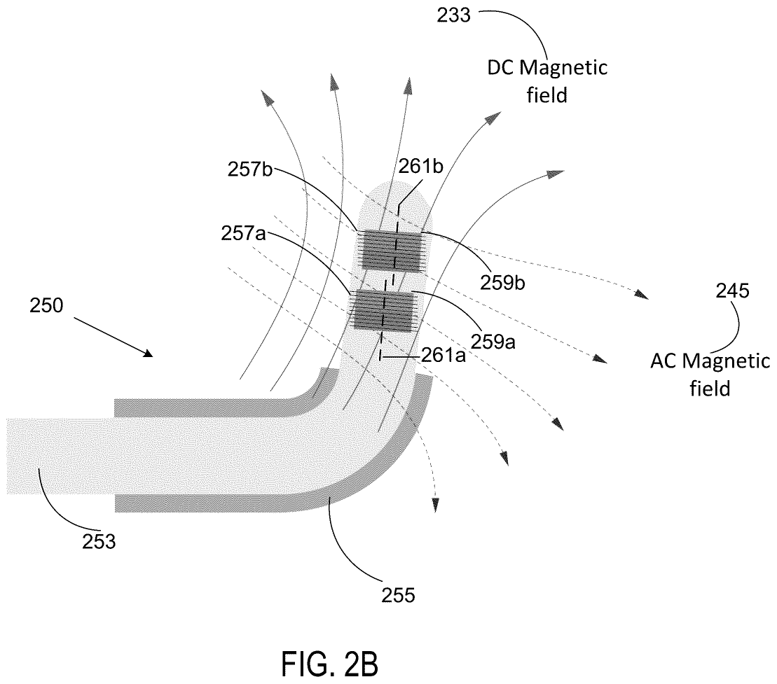

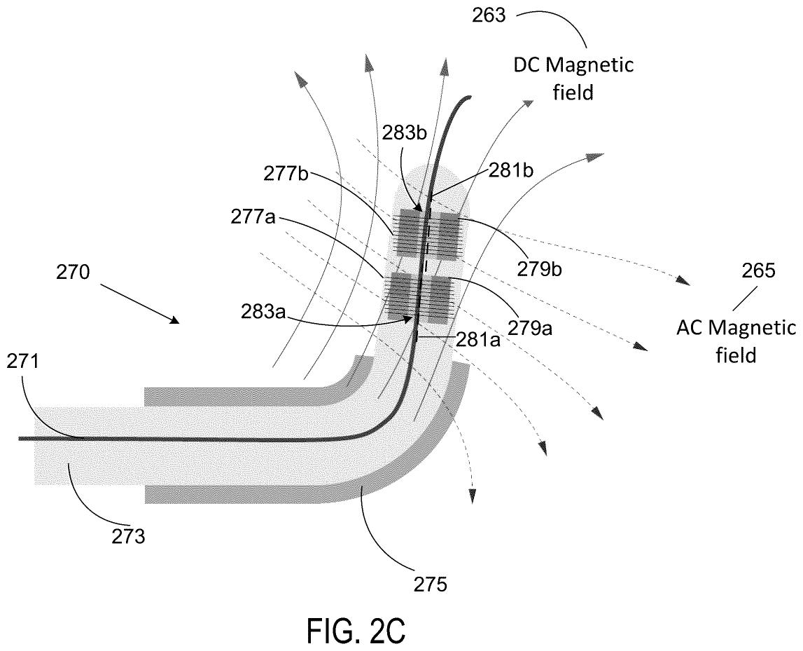

In this example, a catheter 110 is inserted in a patient. The catheter 110 can include one EM sensor (e.g., sensor 227 of FIG. 2A) or two or more EM sensors (e.g., sensors 257a and 257b of FIG. 2B, or sensors 277a and 277b of FIG. 2C). Each of these EM sensors includes coils wound around a permanent magnetic (e.g., permanent magnet 229 of FIG. 2A, permanent magnets 259a and 259b of FIG. 2B, and permanent magnets 279a and 279b of FIG. 2C). This way, the one or more EM sensors can be used to maneuver the device (e.g., the catheter 110) and used to track the catheter 110 by system 100. More specifically, system 100 tracks catheter 110 by measuring and determining the poses of one or more EM sensors in a reference coordinate system 106. Accordingly, the location of the EM sensors relative to a patient can be determined.

To maneuver the device, the system can first determine the location of a catheter, for example, a location in a blood vessel of multiple blood vessels. The location can be tracked using one or more EM sensors as described above. By the interaction force between the external magnetic field and one or more EM sensors, the system can cause the motion of the catheter such that the catheter can be driven by the interaction force to move in a particular direction. The interface force can include repulsive force and attractive force.

A field generator 109 is positioned in the tracking environment. Field generator 109 is configured to generate an external AC magnetic field for tracking locations of a device (e.g., catheter 110, a device or tool including catheter 110). Field generator 109 or another field generator can be configured to generate an external DC magnetic field to maneuver the device (e.g., a tip of the catheter 110 or a tool or device including catheter 110). In the illustrated example, field generator 109 resides beneath the patient. The field generator may be located under a surface that the patient is positioned on, embedded in a table that the patient lays upon, etc., or the field generator 109 may be positioned partially or completely elsewhere in the environment. The field generator 109 is configured to emit electromagnetic fields that are sensed by the accompanying EM sensors (e.g., EM sensors 227, 257a, 257b, 277a, and 277b). In some implementations, the field generator 109 is an NDI Aurora Tabletop Field Generator (TTFG), although other field generator techniques and/or designs can be employed, as known to those skilled in the art.

A fiber optic line 111 (which may or may be guided by a guidewire, not shown) can be extended within catheter 110. The shape of the fiber optic line 111 can be tracked. For example, EM sensors can be measured with respective poses to provide a reference point for fiber optic line 111. The fiber optic line 111 can be tracked relative to one or more of these EM sensors. The fiber optic line 111 can be tracked based on a fiber optic signal. In particular, an interrogator 114 can send and receive fiber optic signals to the tip of the fiber optic line 111 that are indicative of a relative pose (e.g., location and orientation) or a shape of the fiber optic line 111. For example, the measured shape of fiber optic line 111 may be determined based on the fiber optic signals transmitted to and from interrogator 114.

In some implementations, interrogator 114 is an optoelectronic data acquisition system that provides measurements of the light reflected through the optical fiber. Interrogator 114 provides these measurements to the computing device (e.g., the computing device 120) for determining the poses of EM sensors, a shape of the fiber optic line 111, and the corresponding location of the catheter 110.

Optical transducers built into an optical fiber can produce measurements (for example, wavelength measurements) that can be used to estimate pose information along the length of the fiber. Example components, devices, and techniques are described in greater detail in U.S. application Ser. No. 18/151,342, entitled “Electromagnetic Sensor,” filed on Jul. 13, 2023, which is hereby incorporated by reference in its entirety.

Tracking systems are frequently accompanied by computing equipment and displays to process and visualize the measurement data. For example, in a surgical intervention, a surgical tool measured by the tracking system can be visualized with respect to the anatomy marked up with annotations from the pre-operative plan. Another such example may include an X-ray image annotated with live updates from a tracked catheter.

In some cases, system 100 can further include a wireless or wired robot with a magnetic motor. At least a portion of each of one or more EM sensors can be winded around a respective permanent magnet of multiple permanent magnets in the magnetic motor to maneuver and track the robot.

FIGS. 1B and 1C each is a schematic diagram of an example computing device 120 of the electromagnetic system 100 in FIG. 1A and peripheral components coupled to the computing device 120. As shown in FIG. 1B, the computing device 120 shown in FIG. 1A can include a control unit 139 configured to receive and provide data between sensor interface 135, AC magnet field interface 141, and DC magnet field interface 143. For example, control unit 139 is configured to receive input data through AC magnet field interface 141 and generate and send instruction data to interface 141 for controlling AC magnet field generator 145 to generate an external AC magnetic field. The input data from the AC magnet field interface 141 can include user inputs received by interface 141. Similarly, control unit 139 is configured to receive input data through DC magnet field interface 143 and generate and send instruction data to interface 143 for controlling DC magnet field generator 147 to generate an external DC magnetic field. The input data from the DC magnet field interface 143 can include user inputs received by interface 143.

The catheter sensor 133 of FIG. 1B can include one or more EM sensors located in a catheter (e.g., catheter 110 of FIG. 1A). The one or more EM sensors are configured to generate sensor data to be provided to the sensor interface 135. The sensor data can include data representing an induced voltage or an induced magnetization caused by the AC magnetic field generated by the AC magnet field generator 145. The sensor interface 135 can provide the sensor data to control unit 139 for analysis to determine the current pose (e.g., positions, orientations, etc.) of EM sensors, a catheter including the EM sensors, and/or the device or tool that includes the catheter. In some cases, the sensor interface 135 can provide user inputs to the control unit 139 for tracking and/or maneuvering purposes.

The control unit 139 is further configured to generate instructions representing catheter/robotic controls 131 to control the motion of a corresponding catheter and robot. The instructions are generally machine code readable for control engines, and upon executing the catheter/robotic controls 131, the control engines can drive the motion of the corresponding catheter, the robot, or both. The control unit 139 can further generate display data to be presented on display 137. The display data can represent a pose, a location, or both of one or more EM sensors, sensor data collected by a fiber optic line (e.g., fiber optic line 111 of FIG. 1A), etc.

FIG. 1C shows an example computing device similar to that in FIG. 1B and peripheral components coupled to the computing device 120, except that control unit 159 communicates data with an AC magnetic field interface with DC offset 161. In other words, the AC magnet field interface with DC offset 161, upon receiving instructions from control unit 159, is configured to generate both an external AC magnetic field and an external DV magnetic field using a common field generator, e.g., AC and DC magnet fields generator 165.

FIGS. 2A, 2B, and 2C each show a cross-sectional view of a respective example device that includes a catheter and one or more electromagnetic sensors. As shown in FIG. 2A, a device 220 (e.g., to be inserted into a patient) can include catheter 223, which is equivalent to or similar to catheter 111 of FIG. 1A. In some situations, device 220 is a catheter per se. As illustrated in the figures, device 220 is positioned in an external DC magnetic field 213 (represented by solid magnetic lines directed upward) and an external AC magnetic field 215 (represented by dashed lines directed to the right). The external DC magnetic field 213 and external AC magnetic field 215 are substantially orthogonal to each other such that the magnetic fluctuation caused by the AC magnetic field 215 is generally perpendicular to the local directions of the DC magnetic field 213.

An EM sensor coil 227 wrapped at least in part around a permanent magnet 229 is located within the tip of catheter 223. The EM sensor 227 is connected to a signal data line 225 for transmitting sensor data to a computing device (e.g., computing device 110 of FIG. 1A). The permanent magnet 229 can have a magnetization axis 231 that is stable and not susceptible to external magnetic fields 213 and 215. The magnetization axis 231 is determined when the permanent magnet 229 is magnetized. Generally speaking, the magnetization axis 231 is oriented to a direction determined based on the shape of the magnet and the material of the magnet. A magnet with an elongated shape can have a magnetization axis 231 parallel to its elongation axis, yet the material crystallization anisotropy might affect the net direction of the magnetization axis 231. For simplicity and ease of illustration, FIG. 2A represents an elongated magnet 229, and the magnetization axis 231 generally aligns with the elongation axis of magnet 229. However, it is noted that the magnetization axis 231 can point toward directions deviated from the elongation axis, e.g., can be perpendicular to the elongation axis, if desired.

The EM sensor coil 227 generally defines a coil axis (not shown) that is substantially parallel to or aligns with the magnetization axis 231. This way, the EM sensor coil 227 only measures magnetization changes along the magnetization axis 231 and does not detect magnetization changes perpendicular to the magnetization axis 231.

The induced magnetization along the magnetization axis 231 does not push the EM sensor coil 227 into the “saturation” state, and the magnetization changes along the magnetization axis 231 are negligible (ordinarily a quadratic fluctuation). Accordingly, the poses of the EM sensor coil 227 can be accurately measured and determined by the computing system.

As an example, to appreciate the different magnitude ranges in different fields, the AC field can have a magnitude of 0.1 μT, 1 μT, 3 μT, 5 μT, 10 μT, or other suitable values. The DC field at the tip of a catheter accordingly can have a magnitude of 0.1 T, 0.5 T, 1 T, 2 T, or other suitable values. The permanent magnet (e.g., permanent magnet 229) can have a magnitude of 0.5 T, 0.8 T, 1 T, 2 T, 3 T, or other suitable values. As described above, the saturation magnitude in EM sensors depends on the sensor design and can vary between 20 μT and 50 μT. FIG. 2B illustrates a device 250 similar to device 220 of FIG. 2A. However, device 250 includes a sheath or a cover 255 in addition to a catheter 253. The sheath or cover 255 can be insulative, and in some situations, the sheath or cover 255 can be transparent, translucent, or Infrared-transparent.

In addition, device 250 includes two different EM sensor coils 257a and 257b wrapped around permanent magnets 259a and 259b, respectively. Permanent magnets 259a and 259b are positioned sequentially in catheter 273. Each of the permanent magnets 259a and 259b can include a respective magnetization axis 261a, 261b. The magnetization axes 261a and 261b are not necessarily aligned. Similarly, EM sensor coils 257a and 257b each define a coil axis aligning with a corresponding magnetization axis 261a and 261b, respectively.

FIG. 2C illustrates a device 270 similar to device 220 of FIG. 2A and device 250 of FIG. 2B. In addition to sheath 275 and catheter 273, device 270 can include a fiber optic line 271 (similar to the fiber optic line 111 of FIG. 1A) or a guidewire (not shown), or both. The fiber optic line 271 extends through the catheter 273.

As shown in FIG. 2C, device 270 includes two different EM sensor coils 277a and 277b wrapped around two hollow permanent magnets 279a and 279b, respectively. Hollow permanent magnets 279a and 279b are positioned sequentially in catheter 273. Each of the hollow permanent magnets 259a and 259b can include a respective magnetization axis 281a, 281b. Each of the two permanent magnets 279a and 279b respectively defines an inner hole or passage 283a and 283b. The fiber optic line 271 extends through the inner holes 283a and 283b sequentially. EM sensor coils 275a and 257b each define a coil axis aligning with a corresponding magnetization axis 281a and 281b.

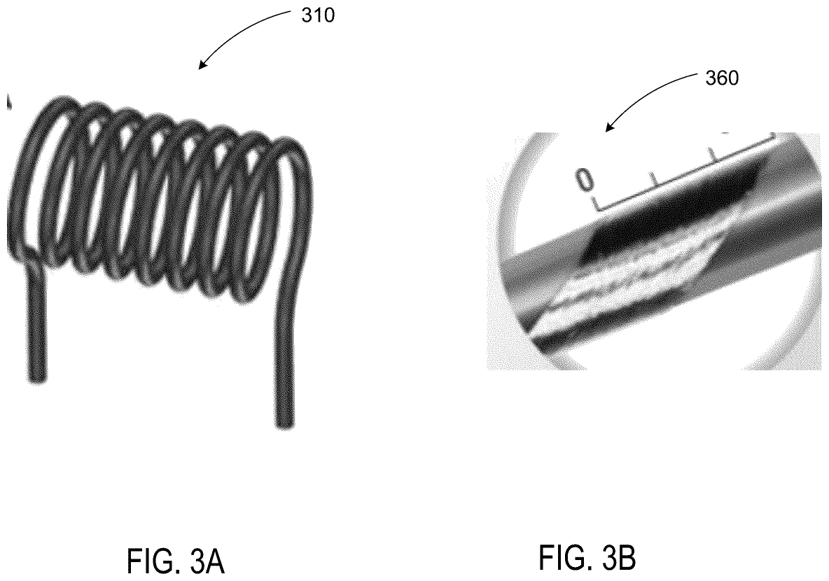

FIGS. 3A and 3B show examples of electromagnetic (EM) sensors 310 and 360. EM sensors 310 and 360 are equivalent to EM sensor coil 227 of FIG. 2A, EM sensor coils 257a and 257b of FIG. 2B, and EM sensor coils 277a and 277b of FIG. 2C.

For example, FIG. 3A illustrates a solenoidal EM sensor 310. The solenoidal EM sensor 310 includes a plurality of substantially orbital turns over a magnet or core (not shown), without any attachment device and/or fixtures being required (e.g., excluding wires attached to EM sensors and/or any potential adhesive, as described in more detail below). For example, no mechanical claptraps holding the EM sensor 310 to catheter 110 of FIG. 1A needs to be employed in the systems and devices described herein. Eliminating such fixtures, which may otherwise offset and/or distort the position of the EM sensor, allows for more accurate shape sensing because the EM sensor 310 is in line with the magnetization axis of a corresponding permanent magnet (e.g., not alongside it). In some implementations, the EM sensor 310 can be wrapped around a hollow magnet, as described above in connection with FIG. 2C.

The coils of the EM sensor 310 can be made from a material such as copper and may be wound on polyimide tubing, which can then be glued on a corresponding permanent magnet.

In some implementations, the coils can be wound on a core of a material that increases sensor sensitivity, such as a ferrite, a mu-metal, a permalloy, or another amorphous metal with high permeability.

As shown in FIG. 3B, the EM sensor 360 may be similar to the solenoidal sensor 310 of FIG. 3A, except the turns of the sensor can be provided at an angle. Due to the particular sensor geometry, a system can provide 6-DOF tracking using two of the EM sensors 360. In implementations in which two EM sensors 360 are used (e.g., to achieve the 6-DOF tracking), two sensors 360 may be rotationally offset from one another, e.g., by 90°.

Referring back to FIG. 1A and FIG. 2C, the operation of system 100 can be controlled by a computing device 120 (e.g., a computer system). In particular, the computing device 120 can be used to interface with the system 100 and cause the locations/orientations of the EM sensors and the fiber optic line 111 to be determined. In this way, the computer system is configured to determine the shape of the fiber optic line extending through the catheter and the position and orientation of one or more solenoidal EM sensors. In particular, the computer system is configured to determine the shape, position, and orientation of a catheter (catheter 110 of FIG. 1A) based on the determined shape of the fiber optic line 111 extending through the catheter and the determined positions and orientations of the EM sensors.

While the systems and devices described herein are related to tracking a catheter using a fiber optic line and one or more EM sensors (as described above in connection with FIG. 2C), similar techniques may also be employed for tracking a guidewire.

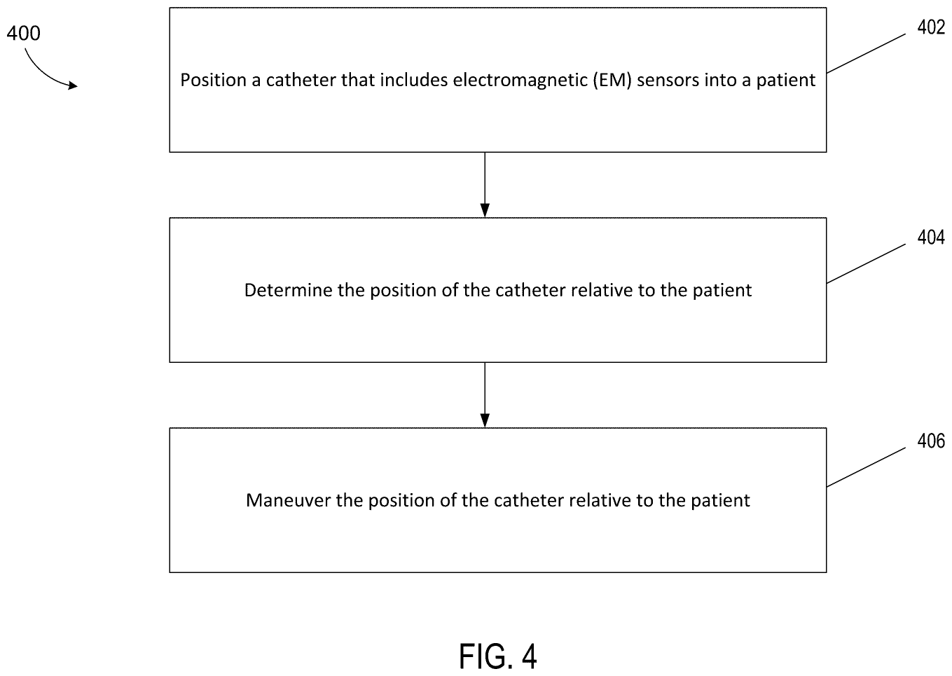

FIG. 4 is a flowchart of operations (process 400) executable by the system of FIG. 1A (e.g., system 100). For convenience, the above-noted procedure or process 400 is described as being performed by a system of one or more computers located in one or more locations. For example, a system, e.g., the electromagnetic system 100 of FIG. 1A, appropriately programmed, can perform process 400.

At step 402, a structure (e.g., a catheter, a guidewire, etc.) is positioned (e.g., within a patient) within one or more external magnetic fields generated by at least one field generator. For example, the structure can include a fiber optic line extending through the structure and one or more electromagnetic (EM) sensors (e.g., two EM sensors). For example, the EM sensors can be located at one end of the structure. For example, the structure can be similar to the catheter 110 of FIG. 1A. The EM sensors can be wrapped around corresponding permanent magnets such that the coil axes of the EM sensors are parallel to the magnetization axes of the corresponding permanent magnets.

At step 404, the system determines the position of the catheter relative to the patient. More specifically, the system can determine the position of the catheter by first determining the shape of a fiber optic line relative to one or more EM sensors. Since the above-described EM sensors are winded around permanent magnets with aligned axes, these EM sensors stay away from the “saturation” states and accordingly can be used to accurately determine the poses of the EM sensors. Once the sensor poses are determined, the shape of the fiber optic line relative to the EM sensors can be determined based on the determined sensor poses. Accordingly, the system can determine an absolute measure of pose (e.g., as opposed to a relative measure of pose) of the fiber optic line. The shape of the fiber optic line relative to the patient can in turn be determined. In some implementations, the reference coordinate system 106 can be used to represent an absolute coordinate system for a portion of the fiber optic line and/or one of the EM sensors.

At step 406, the system maneuvers the position of the catheter relative to the patient. As described above, the system can manipulate the external DC magnetic field to change the position of a device or tool by moving one or more permanent magnets included in the device or tool.

In some cases, the device can include a magnetic motor having two or more permanent magnets. One of the two permanent magnets oscillates in a plane around an oscillating axis, and at least one of the two permanent magnets is winded around by an EM coil having a coil axis parallel to the oscillating axis. The system can move the magnetic motor using the external DC magnetic field and determine the position and orientation of a rotor inside the magnetic motor using the external AC magnetic field.

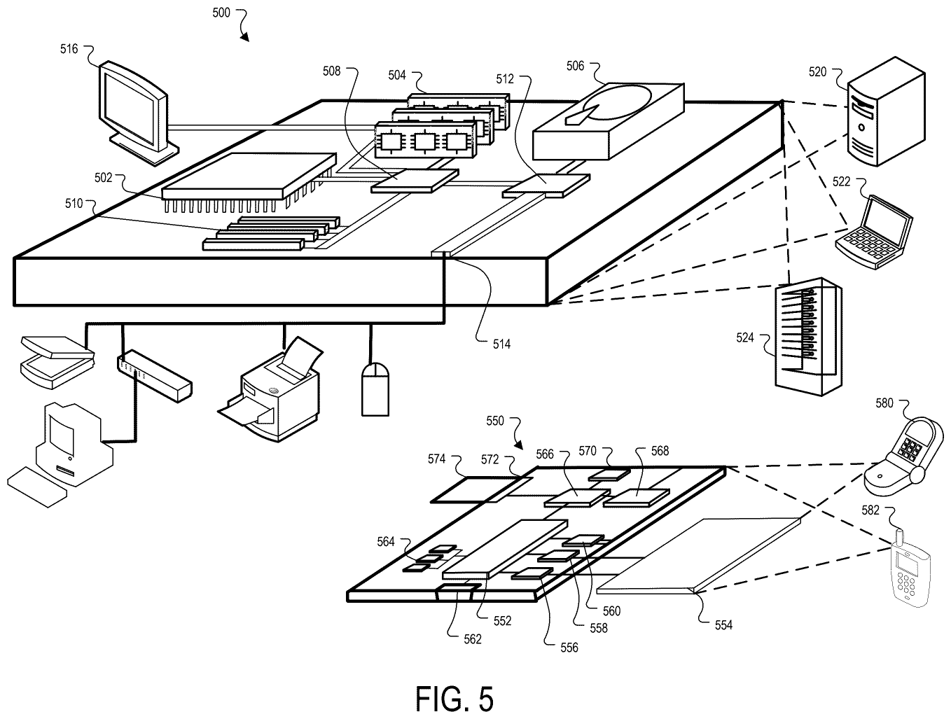

FIG. 5 shows an example computing device 500 and an example mobile computing device 550, which can be used to implement the techniques described herein, e.g., the process 400 described above in connection with FIG. 4. For example, the computing device 500 may be implemented as the computing device 120 of FIG. 1A. Computing device 500 is intended to represent various forms of digital computers, including, e.g., laptops, desktops, workstations, personal digital assistants, servers, blade servers, mainframes, and other appropriate computers. Computing device 550 is intended to represent various forms of mobile devices, including, e.g., personal digital assistants, cellular telephones, smartphones, and other similar computing devices. The components shown here, their connections and relationships, and their functions, are meant to be examples only and are not meant to limit implementations of the techniques described and/or claimed in this document.

Computing device 500 includes processor 502, memory 504, storage device 506, high-speed interface 508 connecting to memory 504 and high-speed expansion ports 510, and low-speed interface 512 connecting to low-speed bus 514 and storage device 506. Each of components 502, 504, 506, 508, 510, and 512, are interconnected using various busses and can be mounted on a common motherboard or in other manners as appropriate. Processor 502 can process instructions for execution within computing device 500, including instructions stored in memory 504 or on storage device 506, to display graphical data for a GUI on an external input/output device, including, e.g., display 516 coupled to high-speed interface 508. In some implementations, multiple processors and/or multiple buses can be used, as appropriate, along with multiple memories and types of memory. In addition, multiple computing devices 500 can be connected, with each device providing portions of the necessary operations (e.g., as a server bank, a group of blade servers, a multi-processor system, etc.).

Memory 504 stores data within the computing device 500. In some implementations, memory 504 is a volatile memory unit or units. In some implementations, memory 504 is a non-volatile memory unit or units. Memory 504 also can be another form of computer-readable medium, including, e.g., a magnetic or optical disk.

Storage device 506 is capable of providing mass storage for computing device 500. In some implementations, storage device 506 can be or contain a computer-readable medium, including, e.g., a floppy disk device, a hard disk device, an optical disk device, a tape device, a flash memory, or other similar solid state memory device, or an array of devices, including devices in a storage area network or other configurations. A computer program product can be tangibly embodied in a data carrier. The computer program product also can contain instructions that, when executed, perform one or more methods, including, e.g., those described above. The data carrier is a computer- or machine-readable medium, including, e.g., memory 504, storage device 506, memory on processor 502, and the like.

High-speed controller 508 manages bandwidth-intensive operations for computing device 500, while low-speed controller 512 manages lower bandwidth-intensive operations. Such allocation of functions is an example only. In some implementations, the high-speed controller 508 is coupled to memory 504, display 516 (e.g., through a graphics processor or accelerator), and to high-speed expansion ports 510, which can accept various expansion cards (not shown). In some implementations, the low-speed controller 512 is coupled to storage device 506 and low-speed expansion port 514. The low-speed expansion port, which can include various communication ports (e.g., USB, Bluetooth®, Ethernet, wireless Ethernet), can be coupled to one or more input/output devices, including, e.g., a keyboard, a pointing device, a scanner, or a networking device including, e.g., a switch or router (e.g., through a network adapter).

Computing device 500 can be implemented in a number of different forms, as shown in FIG. 5. For example, the computing device 500 can be implemented as standard server 520, or multiple times in a group of such servers. The computing device 500 can also be implemented as part of rack server system 524. In addition or as an alternative, the computing device 500 can be implemented in a personal computer (e.g., laptop computer 522). In some examples, components from computing device 500 can be combined with other components in a mobile device (e.g., the mobile computing device 450). Each of such devices can contain one or more computing devices 500, 550, and an entire system can be made up of multiple computing devices 500, 550 communicating with each other.

Computing device 550 includes processor 552, memory 564, and an input/output device including, e.g., display 554, communication interface 566, and transceiver 568, among other components. Device 550 also can be provided with a storage device, including, e.g., a microdrive or other device, to provide additional storage. Components 550, 552, 564, 554, 566, and 568, may each be interconnected using various buses, and several of the components can be mounted on a common motherboard or in other manners as appropriate.

Processor 552 can execute instructions within computing device 550, including instructions stored in memory 564. The processor 552 can be implemented as a chipset of chips that include separate and multiple analog and digital processors. The processor 552 can provide, for example, the coordination of the other components of device 550, including, e.g., control of user interfaces, applications run by device 550, and wireless communication by device 550.

Processor 552 can communicate with a user through control interface 558 and display interface 556 coupled to display 554. Display 554 can be, for example, a TFT LCD (Thin-Film-Transistor Liquid Crystal Display) or an OLED (Organic Light Emitting Diode) display, or other appropriate display technology. The display interface 556 can include appropriate circuitry for driving display 554 to present graphical and other data to a user. The control interface 558 can receive commands from a user and convert them for submission to processor 552. In addition, an external interface 562 can communicate with processor 542, so as to enable near-area communication of device 550 with other devices. External interface 562 can provide, for example, for wired communication in some implementations, or for wireless communication in some implementations. Multiple interfaces also can be used.

Memory 564 stores data within the computing device 550. Memory 564 can be implemented as one or more of a computer-readable medium or media, a volatile memory unit or units, or a non-volatile memory unit or units. Expansion memory 574 also can be provided and connected to device 550 through expansion interface 572, which can include, for example, a SIMM (Single In Line Memory Module) card interface. Such expansion memory 574 can provide extra storage space for device 550, and/or may store applications or other data for device 550. Specifically, expansion memory 574 can also include instructions to carry out or supplement the processes described above and can include secure data. Thus, for example, expansion memory 574 can be provided as a security module for device 550 and can be programmed with instructions that permit secure use of device 550. In addition, secure applications can be provided through the SIMM cards, along with additional data, including, e.g., placing identifying data on the SIMM card in a non-hackable manner.

Memory 564 can include, for example, flash memory and/or NVRAM memory, as discussed below. In some implementations, a computer program product is tangibly embodied in a data carrier. The computer program product contains instructions that, when executed, perform one or more methods. The data carrier is a computer- or machine-readable medium, including, e.g., memory 564, expansion memory 574, and/or memory on processor 552, which can be received, for example, over transceiver 568 or external interface 562.

Device 550 can communicate wirelessly through communication interface 566, which can include digital signal processing circuitry where necessary. Communication interface 566 can provide for communications under various modes or protocols, including, e.g., GSM voice calls, SMS, EMS, or MMS messaging, CDMA, TDMA, PDC, WCDMA, CDMA2000, or GPRS, among others. Such communication can occur, for example, through radio-frequency transceiver 568. In addition, short-range communication can occur, including, e.g., using Bluetooth®, Wi-Fi, or other such transceiver (not shown). In addition, GPS (Global Positioning System) receiver module 570 can provide additional navigation- and location-related wireless data to device 550, which can be used as appropriate by applications running on device 550.

Device 550 also can communicate audibly using audio codec 560, which can receive spoken data from a user and convert it to usable digital data. Audio codec 560 can likewise generate audible sound for a user, including, e.g., through a speaker, e.g., in a handset of device 550. Such sound can include sound from voice telephone calls, recorded sound (e.g., voice messages, music files, and the like), and also sound generated by applications operating on device 550.

Computing device 550 can be implemented in a number of different forms, as shown in FIG. 5. For example, the computing device 550 can be implemented as cellular telephone 580. The computing device 550 also can be implemented as part of smartphone 582, personal digital assistant, or other similar mobile device.

Various implementations of the systems and techniques described here can be realized in digital electronic circuitry, integrated circuitry, specially designed ASICs (application-specific integrated circuits), computer hardware, firmware, software, and/or combinations thereof. These various implementations can include one or more computer programs that are executable and/or interpretable on a programmable system. This includes at least one programmable processor, which can be special or general purpose, coupled to receive data and instructions from and to transmit data and instructions to, a storage system, at least one input device, and at least one output device.

These computer programs (also known as programs, software, software applications, or code) include machine instructions for a programmable processor and can be implemented in a high-level procedural and/or object-oriented programming language, and/or in assembly/machine language. As used herein, the terms machine-readable medium and computer-readable medium refer to a computer program product, apparatus, and/or device (e.g., magnetic discs, optical disks, memory, Programmable Logic Devices (PLDs)) used to provide machine instructions and/or data to a programmable processor, including a machine-readable medium that receives machine instructions.

To provide for interaction with a user, the systems and techniques described herein can be implemented on a computer having a display device (e.g., a CRT (cathode ray tube) or LCD (liquid crystal display) monitor) for presenting data to the user, and a keyboard and a pointing device (e.g., a mouse or a trackball) by which the user can provide input to the computer. Other kinds of devices can be used to provide interaction with a user as well. For example, feedback provided to the user can be a form of sensory feedback (e.g., visual feedback, auditory feedback, or tactile feedback). Input from the user can be received in a form, including acoustic, speech, or tactile input.

The systems and techniques described here can be implemented in a computing system that includes a backend component (e.g., as a data server), or that includes a middleware component (e.g., an application server), or that includes a frontend component (e.g., a client computer having a user interface or a Web browser through which a user can interact with an implementation of the systems and techniques described here), or a combination of such backend, middleware, or frontend components. The components of the system can be interconnected by a form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

In some implementations, the components described herein can be separated, combined or incorporated into a single or combined component. The components depicted in the figures are not intended to limit the systems described herein to the software architectures shown in the figures.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain cases, multitasking and parallel processing may be advantageous.

What is claimed is:

1. A device comprising:
an insertable structure usable in a surgical theater;
one or more permanent magnets positioned at respective locations in the insertable structure, each of the one or more permanent magnets having a magnetization axis; and
one or more electromagnetic sensors, wherein at least a portion of each of the one or more electromagnetic sensors is wrapped around one of the one or more permanent magnets such that a coil axis of the respective electromagnetic sensor substantially aligns with the magnetization axis of the corresponding permanent magnet,
wherein the device is movable according to two different external magnetic fields controlled by a computer system.

2. The device of claim 1, wherein the two different external magnetic fields are generated by at least one magnetic field generator, and the two different external magnetic fields comprise an alternating current (AC) magnetic field and a direct current (DC) magnetic field.

3. The device of claim 2, wherein the device is configured to be located by the computer system using the AC magnetic field, and the device is configured to have the movement initiated by the computer system according to the DC magnetic field.

4. The device of claim 1, wherein at least one of the one or more permanent magnets defines an internal channel.

5. The device of claim 4, wherein the device further comprises a fiber optic line extending through the insertable structure and passing through the internal channel.

6. The device of claim 4, wherein the device further comprises a guidewire extending through the insertable structure and passing through the internal channel.

7. The device of claim 1, wherein one of the one or more electromagnetic sensors has a solenoidal geometry that includes a plurality of windings that include one or more substantially orbital turns.

8. The device of claim 1, wherein the device comprises a magnetic motor, and wherein at least one of the permanent magnets is located inside the magnetic motor.

9. The device of claim 1, wherein one of the one or more permanent magnets oscillates around an oscillating axis, and wherein the coil axis of the electromagnetic sensor substantially aligns with the oscillating axis.

10. The device of claim 1, wherein each of the one or more permanent magnets generates a magnetic field with a magnitude smaller than a magnetic saturation magnitude.

11. A system comprising:
a device, comprising:
an insertable structure usable in a surgical theater,
one or more permanent magnets positioned at respective locations in the insertable structure, each of the one or more permanent magnets having a magnetization axis, and
one or more electromagnetic sensors, wherein at least a portion of each of the one or more electromagnetic sensors is wrapped around one of the one or more permanent magnets such that a coil axis of the respective electromagnetic sensor substantially aligns with the magnetization axis of the corresponding permanent magnet; and
a computer system comprising:
a memory, and
a processor configured to generate a set of instructions that, once executed, control two different external magnetic fields for locating the device and initiating a movement of the device.

12. The system of claim 11, wherein the two different external magnetic fields are generated by at least one magnetic field generator, and the two different external magnetic fields comprise an alternating current (AC) magnetic field and a direct current (DC) magnetic field.

13. The system of claim 12, wherein the device is configured to be located by the computer system using the AC magnetic field, and the device is configured to have the movement initiated by the computer system according to the DC magnetic field.

14. The system of claim 11, wherein at least one of the one or more permanent magnets defines an internal channel.

15. The system of claim 14, wherein the device further comprises a fiber optic line extending through the insertable structure and passing through the internal channel.

16. The system of claim 14, wherein the device further comprises a guidewire extending through the insertable structure and passing through the internal channel.

17. The system of claim 11, wherein one of the one or more electromagnetic sensors has a solenoidal geometry that includes a plurality of windings that include one or more substantially orbital turns.

18. The system of claim 11, wherein the device comprises a magnetic motor, and wherein at least one of the permanent magnets is located inside the magnetic motor.

19. The system of claim 11, wherein one of the one or more permanent magnets oscillates around an oscillating axis, and wherein the coil axis of the electromagnetic sensor substantially aligns with the oscillating axis.

20. The system of claim 11, wherein each of the one or more permanent magnets generates a magnetic field with a magnitude smaller than a magnetic saturation magnitude.
